(19) United States
(12) Patent Application Publication
(10) Pub. No.: US 2007/0213827 A1
(43) Pub. Date: Sep. 13, 2007

(54) Hardened calcium phosphate cement bone implants

(76) Inventor: Yves P. Arramon, Sunnyvale, CA (US)

Correspondence Address: MEYERTONS, HOOD, KIVLIN, KOWERT & GOETZEL, P.C., P.O. BOX 398, AUSTIN, TX, 78767-0398, US

(21) Appl. No.: 11/529,565

(22) Filed: Sep. 28, 2006

Related U.S. Application Data
(60) Provisional application No. 60/721,299, filed on Sep. 28, 2005.

(51) Int. Cl. A61F 2/44; A61F 2/28

(52) U.S. Cl. 623/17.11; 623/23.5

(57) ABSTRACT

In some embodiments, an implant may include a porous component and a load bearing component at least partially surrounding the porous component. The porous component has a porosity that is greater than the porosity of the load bearing component. One or more protrusions may be present on the load bearing component. The porous component and/or a load bearing component may be at least partially composed of calcium phosphate.

### CROSS-REFERENCE TO RELATED APPLICATIONS

This application claims the right of priority under 35 U.S.C. §119(e) to U.S. Provisional Application No. 60/721,299 entitled “HARDENED CALCIUM PHOSPHATE CEMENT BONE IMPLANTS” by Arramon, et al., filed Sep. 28, 2005, which is incorporated by reference in its entirety as though fully set forth herein.

### BACKGROUND OF THE INVENTION

1. Field of the Invention

The present invention is generally relates to calcium phosphate prosthetic bone implants incorporating bioactive compositions and methods of making same. More particularly, the invention relates to the use of calcium phosphate prosthetic bone implants coated with calcium phosphate layers as delivery vehicles for bioactive compositions.

2. Description of the Relevant Art

Prosthetic bone implants and bone substitute materials are commonly used in medical procedures carried out for plastic or reconstructive surgery, orthopedic or dental surgery, dental implantology, and to treat a number of conditions involving calcified tissues. Such procedures include correction of bone defects resulting from trauma or surgery (e.g., following excision of a tumor), correction of a congenital malformation involving a calcified structure. Ideally, prosthetic bone implants would be made from calcified autogenic bone material. However, the availability of autogenic bone and the potential of allogenic bone for initiating immunologic rejections makes the use of natural bone grafts impractical and expensive for widespread use.

Synthetic biocompatible (e.g., toxicologically and immunologically inert) calcium phosphate ceramics can be manufactured inexpensively and in large scale. The use of calcium phosphate ceramic materials, and in particular hydroxyapatite, as prosthetic implants has been hampered however, by the combined observations that unsintered calcium phosphate materials lack sufficient compressive strength and load bearing capacity to be of substantial benefit as a bone prostheses. Additionally, sintered calcium phosphate ceramics, while able to bear higher compressive forces, are typically too brittle, and not of sufficient porosity to enable cellular and vascular infiltration of the implant to the extent necessary to promote remodeling and resorbtion of the implant. Mechanical and structural deficiencies notwithstanding, bioceramic hydroxyapatites (HAp) have been widely employed in prosthetic applications for several years. The suitability of HAp as a prosthetic implant material stems from the facts that it is relatively easy and cheap to manufacture, is nontoxic, and appears to attach well to calcified tissues. Moreover HAp has the advantageous property of being able to conduct bone apposition, the bone remodeling process that initially establishes fixation of an uncemented implant to adjacent bone.

Metal-based implants or endoprostheses have been used for many decades in clinical dentistry and orthopedic surgery. Titanium and its alloys are especially popular due to their excellent mechanical properties and ease of handling during surgery. Furthermore, they are highly biocompatible with the bony tissue compartment. Metal or metal composite implants can be rendered more biocompatible by coating the surface thereof with biocompatible materials such as crystalline HAp, which has the further advantage of being able to act as a pharmaceutical carrier medium. However, HAp crystals are not easily grown on the surface of metallic implants, particularly under the physiological conditions required to retain biological activity of some bioactive agents used in orthopedic applications. Techniques have been developed whereby metal implants are first coated with one or more layers of amorphous calcium phosphate. The amorphous calcium phosphate layers prime the surface of the implant and act as seeds that promote precipitation of a layer of ordered HAp crystals. Moreover, under these conditions, crystalline HAp can be precipitated in aqueous solutions under physiological conditions, allowing the co-precipitation of polypeptide growth factors in the precipitating solutions. The elution profile of such co-precipitated factors is much slower than implants coated by more traditional methods.

The publication by Liu et al. entitled “Osteoinductive Implants: The Mise-en-scène for Drug-Bearing Biomimetic Coatings” appearing in March 2004 in Vol. 32, pp. 398-406 of Annals of Biomedical Engineering describes titanium metal alloy implants coated with amorphous calcium phosphate and crystalline HAp, and methods of making same. The biocompatible implants described by Liu can exhibit high compressive strength, but are not bioresorbable. Moreover, the requirement for the deposition of multiple calcium phosphate layers, and thus multiple surface treatments, adds layers of complexity and requires additional quality control measures.

U.S. Patent Application Serial No. 2005/0169964 by Zitelli et al. entitled “Antibiotic calcium phosphate coating” describes a method for applying calcium phosphate surface layers containing therapeutic agents such as antibiotics or bone proteins to a metallic prosthesis.

U.S. Patent Application Serial No. 2005/0170070 by Layrolle et al. entitled “Method for applying a bioactive coating on a medical device” describes ceramic coatings containing bioactive agents formed on the surfaces of medical devices made of inorganic, metallic or organic materials, and methods and systems for making same. The coatings are deposited on the implant surface by passing the implant through a stream of a coating solution in a reactor system.

U.S. Patent Application Serial No. 2005/0031704 by Ahn et al. entitled “Tricalcium phosphates, their composites, implants incorporating them, and method for their production” describes bioceramics, particularly tricalcium phosphate bioceramics, composites incorporating these materials, and methods for their production. The surface of a calcium phosphate powder such as TCP or hydroxyapatite may contain therapeutic compositions (e.g., nucleic acids, proteins, or antibiotics) for drug delivery.

U.S. Patent Application Serial No. 2005/0106260 by Constanz et al. entitled “Calcium phosphate cements comprising an osteoclastogenic agent” describes injectable calcium phosphate cement pastes that include osteoclastogenic agents.

U.S. Patent Application Serial No. 20050119761 by Matsumoto et al. entitled “Porous calcium phosphate ceramic and method for producing same” describes sintered calcium phosphate ceramics with macroporosity for use in medical applications. The ceramic is capable of binding polypeptides.

U.S. Patent Application Serial No. 20040091544 by Ruff et al. entitled “Coated dibasic calcium phosphate” describes dibasic calcium phosphate coatings as pharmaceutical carriers for sustained release of orally administered peptides.

U.S. Patent Application Serial No. 20020156529 by Lin et al. entitled “Surface-mineralized spinal implants” describes spinal implants with mineralized bioactive surfaces chemically coated on the implant. The coatings are non-hydroxyl containing carbonated calcium phosphate bone mineral nanocrystalline apatite less than about 1 μm in size.

U.S. Patent Application Serial No. 20030170378 by Wen et al. entitled “Novel materials for dental and biomedical application” describes apatite-like calcium phosphate complexes for use in biomedical and dental applications. The complexes may include apatite, octacalcium phosphate crystals, or mixtures thereof. The complexes are nucleated on titanium metal surfaces by placing a titanium substrate in a supersaturated calcifying solution containing native or purified recombinant amelogenins, which modulate apatite crystal growth to mimic in vivo apatite crystal formation.

U.S. Pat. No. 6,808,561 to Genge et al. entitled “Biocompatible cement containing reactive calcium phosphate nanoparticles and methods for making and using such cement” describes cement powders that contain reactive tricalcium phosphate nanoparticles and methods of making same.

U.S. Pat. No. 6,730,129 to Hall entitled “Implant for application in bone, method for producing such an implant, and use of such an implant” describes bone implants made of a biocompatible material such as titanium, and having one or more calcium phosphate coatings comprising a bone-growth-stimulating substance that initiates and/or stimulates bone growth. The coating is applied at least to surface parts of the unit cooperating with the bone. A method of producing the implant is also provided.

U.S. Pat. No. 5,876,452 to Athanasiou et al., entitled “Biodegradable implant” describes biodegradable, porous, polymeric implant materials that provide substantially continuous release of bioactive agent during in vivo use. Bioactive agent is initially released in amounts that are less than degradation rate of polymer, thereby promoting migration of cells into material. Later larger amounts of bioactive agent are released, thereby promoting differentiation of cells. Method of making material includes steps of applying vacuum temperature and consession to form pores. Implant material may be adapted for one phase implant (e.g., for bone or cartilage) or for two phase layered implant (e.g., for cartilage layer on top of bone layer).

U.S. Pat. No. 4,563,489 to Urist entitled “Biodegradable organic polymer delivery system for bone morphogenetic protein” describes biodegradable polylacetic acid polymer delivery system for delivery of bone morphogenic protein (BMP) to induce formation of new bone in viable tissue. The delivery composition is substantially pure BMP in combination with a biodegradable polylacetic acid polymer and it is prepared by admixing the BMP with the biodegradable polymer. The composition is implanted in viable tissue where the BMP is slowly released and induces formation of new bone.

The aforementioned prior art references are incorporated by reference as though fully set forth herein.

### SUMMARY OF THE INVENTION

In some embodiments, a bone implant may include one or more porous components at least partially surrounded by a load bearing component. One or more porous components may have a porosity greater than the porosity of the load bearing component. A porous component may include an opening. The opening in the porous component may extend through the porous component. One or more additives may be positioned in the opening in the porous component.

In an embodiment, a load bearing component may at least partially surround a porous component. A load bearing component may include one or more protrusions on a surface. A protrusion may retain an implant in a spine. A load bearing component may include one or more channels.

In some embodiments, a porous component and/or a load bearing component may be at least partially composed of calcium phosphate. A porous component and/or a load bearing component may be formed from calcium phosphate particles with a diameter less than 100 μm and greater than 0.05 μm. The calcium phosphate particles may include whiskers with a width less than 100 nm and greater than 1 nm. The calcium phosphate particles may include whiskers with a length less than 1000 nm and greater than 1 nm.

### BRIEF DESCRIPTION OF THE DRAWINGS

Other objects and advantages of the invention will become apparent upon reading the following detailed description and upon reference to the accompanying drawings in which:

FIG. 1 depicts an embodiment of a bone implant with two protrusions;

FIG. 2A depicts an embodiment of a bone implant with four protrusions;

FIG. 2B depicts an embodiment of a bone implant with protrusions that do not extend beyond a surface of a porous component;

FIG. 3 depicts an embodiment of a bone implant with two protrusions;

FIG. 4 depicts a side view of an embodiment of a bone implant;

FIG. 5 depicts an embodiment of a bone implant with four protrusions;

FIG. 6 depicts an embodiment of a bone implant with a porous component that extends beyond a surface of a load bearing component;

FIG. 7 depicts a cross-sectional view of an embodiment of a bone implant with porous components in channels of a load bearing component;

FIGS. 8-10 depict embodiments of portions of a bone implant with porous components in a load bearing component;

FIG. 11 depicts a cross-sectional view of an embodiment of a bone implant with a porous component on a surface of a load bearing component;

FIG. 12 shows S.E.M. images (taken at 10,000-fold magnification) of implant surfaces having nanoporous nanocrystalline calcium phosphate material made by soaking the implant in; FIG. 12A) Hank's Balanced Salt Solution (with Ca and Mg) for 3 days; and FIG. 12B) phosphate buffered saline for 5 days.

While the invention is susceptible to various modifications and alternative forms, specific embodiments thereof are shown by way of example in the drawings and will herein be described in detail. It should be understood, however, that the drawing and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

Definitions

In order to facilitate understanding of the invention, a number of terms are defined below. It will further be understood that, unless otherwise defined, all technical and scientific terminology used herein has the same meaning as commonly understood by practitioners of ordinary skill in the art to which this invention pertains.

As used herein, a material, composition or object that is “bioresorbable,” generally refers to a biocompatible material, composition or object that has the ability to be gradually integrated into a host. When used in the context of the subject prosthetic bone implants, the term generally refers to the ability of at least a portion of the prosthetic bone implant to gradually be replaced by natural bone, such replacement typically occurring naturally by the physiological process of bone remodeling. Thus, in the context of the presently described embodiments, the term “bioresorbable” is meant to include any material or process that is receptive to or typically associated with bone remodeling, including but not limited to osteoblast and osteoclast activity, deposition and/or mineralization of new bone matrix, vascular and cellular infiltration and tissue ingrowth.

As used herein, the term “unsintered,” when used in the context of the subject prosthetic bone implants, generally refers to a prosthetic bone implant that is made from a hardened calcium phosphate cement and that has not undergone a high temperature sintering step. While sintered calcium phosphate ceramics exhibit relatively high tensile strength and biocompatibility, they typically are less porous, and as a result are generally not bioresorbable. Thus, unsintered calcium phosphate cement articles retain their porosity, and are therefore more bioresorbable than sintered calcium phosphate ceramics. Included within the term “unsintered” are those bioresorbable calcium phosphate articles or cements that have been treated at a temperature up to 750° C., up to 500° C., up to 200° C., or up to 50° C.

As used herein, the terms “cortical portion” or “cortical,” when used in the context of the subject prosthetic bone implants, generally refers to a portion of the prosthetic bone implant that functions in a load-bearing capacity and whose function and structure are substantially similar to that of naturally occurring cortical or compact bone.

As used herein, the terms “cancellous portion,” or “cancellous” when used in the context of the subject prosthetic bone implants, generally refer to portions of the subject prosthetic bone implants that are more porous than the cortical portions, and whose structure and function of which are substantially similar to that of naturally occurring trabecular or spongy bone. Due to its high degree of porosity, a cancellous portion has a relatively high surface area and can support tissue ingrowth and infiltration of body fluids and cells. A cancellous portion may also increase the wicking profile of a prosthetic bone implant.

As used herein, the term “apatite” generally refers to a group of phosphate minerals, (typically to hydroxyapatite, fluorapatite, and chlorapatite) having the general chemical formula Ca₅(PO₄)₃X, where X is OH, F, or Cl. The term “hydroxyapatite” or “HAp” as used herein, generally refers to a form of apatite with the formula Ca₅(PO₄)₃(OH), but is more typically represented as Ca₁₀(PO₄)₆(OH)₂ to denote that the crystal unit cell comprises two molecules. Hydroxylapatite is the hydroxylated member of the complex apatite group. The hardness of hydroxyapatite may be altered by replacing the OH ion with other anions (e.g., fluoride, chloride or carbonate). Additionally, HAp has a relatively high affinity for peptides, making it an ideal carrier for the delivery and sustained release of polypeptides over long periods of time in situ. Materials that are referred to herein as “apatitic,” are generally those materials that have apatite as the major phase.

As used herein, the term “crystalline” is an art-recognized term that is used to describe a mineral composition having relatively a well-defined crystal structure, with a unique arrangement of atoms within the component crystals. There are at least 7 art-recognized crystals systems. Pure hydroxyapatite typically crystallizes in the hexagonal crystal system, although alternate crystal structures may be realized by altering the composition of the mineral.

As used herein, the term “amorphous,” when used in the context of mineral compositions, generally refers to a relatively unstructured, non-crystalline form of a mineral that is capable of acting as a seed and support for the growth of crystals thereon.

As used herein, the term “bioactive composition” generally refers to a composition that is capable of inducing or affecting an action in a biological system, e.g. by inducing or affecting a therapeutic or prophylacetic effect, an immune response, tissue growth, cell growth, cell differentiation or cell proliferation. A bioactive composition may include a pharmaceutical delivery vehicle. The delivery vehicle would typically be optimized to stably accommodate an effective dosage of one or more compounds having biological activity. The determination of the effective dose of a bioactive compound that should be included in a bioactive composition to achieve a desired biological response is dependent on the particular compound, the magnitude of the desired response, and the physiological context of the composition. Such determinations may be readily made by an ordinary practitioner of the pharmaceutical arts. Components of bioactive compositions may include growth factors, bone proteins, analgesics, antibiotics, or other pharmacologically active compounds.

As used herein, the term “osteoinductive,” when used in the context of a bioactive composition, generally refers to a composition that induces and/or supports the formation, development and growth of new bone, and/or the remodeling of existing bone. An osteoinductive composition typically includes one or more osteogenic agents. An “osteogenic agent,” as used herein, is an agent that can elicit, facilitate and/or maintain the formation and growth of bone tissue. Many osteogenic agents function, at least in part, by stimulating or otherwise regulating the activity of osteoblast and/or osteoclasts. Exemplary osteogenic agents include certain polypeptide growth factors, such as, osteogenin, Insulin-like Growth Factor (IGF)-1, TGF-β1, TGF-β2, TGF-β3, TGF-β4, TGF-β5, osteoinductive factor (OIF), basic Fibroblast Growth Factor (bFGF), acidic Fibroblast Growth Factor (aFGF), Platelet-Derived Growth Factor (PDGF), vascular endothelial growth factor (VEGF), Growth Hormone (GH), osteogenic protein-1 (OP-1) and any one of the many known bone morphogenic proteins (BMPs), including but not limited to BMP-1, BMP-2, BMP-2A, BMP-2B, BMP-3, BMP-3b, BMP-4, BMP-5, BMP-6, BMP-7, BMP-8, BMP-8b, BMP-9, BMP-10, BMP-11, BMP-12, BMP-13, BMP-14, BMP-15. An osteoinductive composition may include one or more agents that support the formation, development and growth of new bone, and/or the remodeling thereof. Typical examples of compounds that function in such a supportive manner include, though are not limited to, extracellular matrix-associated bone proteins (e.g., alkaline phosphatase, osteocalcin, bone sialoprotein (BSP) and osteocalcin in secreted phosphoprotein (SPP)-1, type I collagen, fibronectin, osteonectin, thrombospondin, matrix-gla-protein, SPARC, alkaline phosphatase and osteopontin).

As used herein, the term “growth factor” generally refers to a factor, typically a polypeptide, that affects some aspect of the growth and/or differentiation of cells, tissues, organs, or organisms.

As used herein, the term “bone morphogenic protein,” or “BMP” generally refers to a group of polypeptide growth factors belonging to the TGF-β superfamily. BMPs are widely expressed in many tissues, though many function, at least in part, by influencing the formation, maintenance, structure or remodeling of bone or other calcified tissues. Members of the BMP family are potentially useful as therapeutics. For example, BMP-2 has been shown in clinical studies to be of use in the treatment of a variety of bone-related conditions.

As used herein, the term “bone protein” generally refers to a polypeptide factor that supports the growth, remodeling, mineralization or maintenance of calcified tissues. Bone proteins are typically components of, or associate with cells and structures that form extracellular matrix structures. Typical examples of bone proteins may include, though are not limited to, alkaline phosphatase, osteocalcin, bone sialoprotein (BSP) and osteocalcin in secreted phosphoprotein (SPP)-1, type I collagen, type IV collagen, fibronectin, osteonectin, thrombospondin, matrix-gla-protein, SPARC, alkaline phosphatase and osteopontin.

As used herein, the term “antibiotic” generally refers to a naturally occurring, synthetic or semi-synthetic chemical substance that is derivable from a mold or bacterium that, when diluted in an aqueous medium, kills or inhibits the growth of microorganisms and can cure or treat infection.

As used herein, the term “analgesic” is used in reference to a pharmacologically active agent or composition that alleviates pain without causing loss of consciousness.

As used herein, the term “polypeptide” generally refers to a naturally occurring, recombinant or synthetic polymer of amino acids, regardless of length or post-translational modification (e.g., cleavage, phosphorylation, glycosylation, acetylation, methylation, isomerization, reduction, farnesylation, etc . . . ), that are covalently coupled to each other by sequential peptide bonds. Although a “large” polypeptide is typically referred to in the art as a “protein” the terms “polypeptide” and “protein” are often used interchangeably. The term “portion”, as used herein in the context of a polypeptide (as in “a portion of a given polypeptide/polynucleotide”) generally refers to fragments of that molecule. The fragments may range in size from three amino acid or nucleotide residues to the entire molecule minus one amino acid or nucleotide. Thus, for example, a polypeptide “comprising at least a portion of the polypeptide sequence” encompasses the polypeptide defined by the sequence, and fragments thereof, including but not limited to the entire polypeptide minus one amino acid.

As used herein, the term “whisker,” when used in the context of a calcium phosphate materials, generally refers to thin, needle-like calcium phosphate crystals that form on the surface of calcium phosphate particles after subjecting the particles to specific processes as defined below.

As used herein, the term “interconnected porosity” generally refers to pores or cavities in the body or matrix of the subject prosthetic bone implants whose pores are coupled to each other and form a continuous network of pores capable of conveying liquids or gases, or materials dissolved therein. Typically, the amount of interconnected porosity of the subject implants is related to the bioresorbability.

As used herein, the term “pore throat diameter” generally refers to the size or diameter of the openings between adjacent pores, or between a pore and the implant surface.

As used herein, the term “non-dispersible,” when used in the context of the presently described calcium phosphate cements, generally refers to a physical property of the cement whereby a paste made by combining the cement powder with a setting liquid resists dispersion in an aqueous environment. The ability of a calcium phosphate cement paste to resist dispersion may be related to the surface structure of its constituent particles.

As used herein, the term “nanocrystalline” generally refers to a ceramic material whose polycrystalline grain structure is reduced from the micron range to the nanometer range. The surface of a nanocrystalline ceramic has physico-chemical properties that distinguish its polycrystalline counterpart and may make it more receptive to binding certain molecules and ions. Nanocrystalline calcium phosphate may be formed through the crystallization of amorphous calcium phosphate.

As used herein, the term “nanoporous” generally refers to a porous material (i.e. a calcium phosphate ceramic) whose average pore diameter is in the nanometer range (typically between 1 to 1000 nm).

As used herein, the term “wicking” generally refers to the ability of a porous calcium phosphate article to convey liquid by capillary action.

The following descriptions are directed to porous, bioresorbable calcium phosphate prosthetic bone implants having high compressive strength (>50 MPa) that may also function as pharmaceutical carriers for bioactive compositions. The presently described embodiments are further directed methods of making same. The implants will typically be made from hardened apatitic calcium phosphate cements (CPC).

Calcium Phosphate Cements

Calcium phosphate cements, as well as the methods used in the manufacture thereof, suitable for use with the presently described embodiments generally include, without limitation, those calcium phosphate cement compositions disclosed in U.S. Pat. Nos. 6,379,453 and 6,840,995 to Lin et al., entitled “PROCESS FOR PRODUCING FAST SETTING, BIORESORBABLE CALCIUM PHOSPHATE CEMENT”; U.S. Pat. No. 7,094,282 to Lin et al., entitled “CALCIUM PHOSPHATE CEMENT, USE AND PREPARATION THEREOF”; U.S. Pat. No. 6,960,249 to Lin et al., entitled “TETRACALCIUM PHOSPHATE (TTCP) HAVING CALCIUM PHOSPHATE WHISKER ON SURFACE”; U.S. Pat. No. 7,066,999 to Lin et al., entitled “PROCESS FOR PRODUCING FAST-SETTING BIORESORBABLE CALCIUM PHOSPHATE CEMENT”; U.S. Patent Appl. Publ. No. 2004/0175320 by Lin et al., entitled “TETRACALCIUM PHOSPHATE (TTCP) HAVING CALCIUM PHOSPHATE WHISKER ON SURFACE AND PROCESS FOR PREPARING THE SAME”; U.S. Patent Appl. Publ. No. 2005/0069479 by Lin et al., entitled “METHOD OF INCREASING WORKING TIME OF TETRACALCIUM PHOSPHATE CEMENT PASTE”; U.S. Patent Appl. Publ. Nos. 2005/0271741; 2005/0271740; 2005/0271742; and 2005/0268819 by Lin et al., entitled “CALCIUM PHOSPHATE CEMENT, USE AND PREPARATION THEREOF”; U.S. Patent Appl. Publ. Nos. 2005/0279252; 2005/0268820; and 2005/0268821 by Lin et al., entitled “TETRACALCIUM PHOSPHATE (TTCP) HAVING CALCIUM PHOSPHATE WHISKER ON SURFACE”; U.S. Patent Appl. Publ. Nos. 2005/0274287; 2005/0274286 and 2005/0274282 by Lin et al., entitled “TETRACALCIUM PHOSPHATE (TTCP) HAVING CALCIUM PHOSPHATE WHISKER ON SURFACE AND PROCESS FOR PREPARING THE SAME”;U.S. Patent Appl. Publ. Nos. 2005/0274288; 2005/0274289; 2006/0011100; and 2006/0011099 by Lin et al., entitled “PROCESS FOR PRODUCING FAST-SETTING BIORESORBABLE CALCIUM PHOSPHATE CEMENT”; and U.S. Patent Appl. Publ. No. 2005/0279256 by Lin et al., entitled “METHOD OF INCREASING WORKING TIME OF TETRACALCIUM PHOSPHATE CEMENT PASTE.” The above-cited patents and patent applications are commonly owned with the present invention and the contents thereof are hereby incorporated by reference in their entirety as though fully set forth herein. Calcium phosphate cements may be formed from acidic calcium phosphates (e.g., calcium phosphates having a calcium to phosphorous ratio of less than 1.33), basic calcium phosphates (e.g., calcium phosphates having a calcium to phosphorous ratio of greater than 1.33) or combinations of acidic and basic calcium phosphates. The presently described CPCs may optionally include one or more bioactive compositions dispersed or dissolved therein, such as are described in detail below.

Particularly suited to the presently described embodiments are CPC formulations that include calcium phosphate particles having whiskers on the surface of the particles, such as are disclosed in the above-cited references. Without being bound to any specific theories or mechanisms, the surface whiskers described in these references increase the surface area of cement particles and allow for improved cementing reactions to occur, resulting in hardened materials having improved compressive strength. Additionally, and by virtue of their ability to form interlocking complexes with the whiskers of adjacent particles, surface whiskers advantageously allow a CPC paste that includes said particles to be substantially non-dispersive in aqueous solutions. Thus, these non-dispersive pastes are well suited to therapeutic applications in which a CPC paste is injected to a site the body of the subject where there exists the possibility that the paste would be washed away by body fluids prior to the hardening thereof.

In an embodiment, whiskers comprising TTCP may be formed on the surface of TTCP particles by soaking the particles in an aqueous phosphate solution having basic pH. Without being bound by any particular theory or mechanism of action, crystalline TTCP that is exposed to alkaline solutions (typically at a pH of about 8.0) for a period of several minutes (e.g. typically bout 5 minutes), may result in the dissolution of a portion of the calcium phosphate material into the aqueous surrounding. The loss of the calcium phosphate material into the aqueous solution may contribute to the formation of TTCP crystals on the surface of TTCP particles (e.g. etching). Typically, the etching seen during formation of the whiskers described above and in the above-cited references follows the grain boundaries of the calcium phosphate crystals.

In some embodiments, calcium phosphate cements used with the present invention may be prepared in accordance with an alternate procedure set forth below.

Tetracalcium Phosphate (TTCP) Synthesis:

In an embodiment, Dibasic Calcium Phosphate, Anhydrate (DCPA; CaHPO₄) or alternatively Calcium pyrophosphate (Ca₂P₂O₇) may be combined with calcium carbonate (CaCO₃) such that the Ca/P molar ratio is >2.0. By way of non-limiting example, 1008.73 grams of dibasic calcium phosphate, anhydrate may be combined with 816.270 grams of calcium carbonate such that the Ca/P molar ratio is 2.1. In some embodiments, it may desirable that the amount of magnesium contamination in both powders is controlled. A typical acceptable contamination level of magnesium in DCPA is less than about 2000 ppm magnesium (by weight) and more preferably about 500-1000 ppm magnesium (by weight). The amount of magnesium may be determined using, e.g., spectrometric methods routinely performed in the art such as inductively coupled plasma mass spectrometry. A typical contamination level in calcium carbonate is approximately 2000 to 3000 ppm magnesium. These initial magnesium levels in the raw materials yield a magnesium level in the final product of approximately 1000 to 2000 ppm. Greater than about 2000 ppm magnesium is generally undesirable. The effects and implications of less than 1000 ppm magnesium are uncertain at this time.

The powders are blended in an organic solvent, e.g., an alcohol (L/S=0.6 ml/gm). The excess alcohol is removed such e.g., by vacuum filtration and/or evaporation in a drying oven. The dried powder is lightly broken up, such as in a bowl with a spatula or pestle, placed in a crucible and fired in a furnace. In certain embodiments, the typical firing profile when calcium pyrophosphate is used is immediate ramping to 100° C. at 20° C./minute with a 0 to 4 hour dwell time followed by a temperature ramp at 5° C./minute up to 800° C., then ramping at 10° C./minute up to 1200° C. Then the temperature is ramped at 4° C./minute up to 1400° C. and allowed to soak for 12 hours. Alternatively, when DCPA is used, in order to accommodate the loss of hydrogen and oxygen as water at lower temperatures, the filled crucibles are fired in a furnace with a temperature profile which ramps up to 100° C. immediately at 20° C./minute and dwells for 0 to 4 hours. Then the temperature is ramped at 5° C./minute up to 600° C., then ramped at 10° C./minute up to 1200° C. Then the temperature is ramped at 4° C./minute up to 1400° C. and allowed to soak for 12 hours. In either embodiment, after the soak, the furnace is allowed to cool to 1000° C. at the natural cooling rate of the furnace (˜10°/min). When the temperature drops below about 1000° C. the furnace door is opened to speed cooling to room temperature.

The cooled tetracalcium phosphate cakes are crushed to <500 microns then milled to a bimodal distribution where 50% of the particles are below approximately 7 to 11 microns. Typical final milling can be performed using a ball mill at 60 r.p.m. in approximately 45 to 60 minutes.

Fine Dibasic Calcium Phosphate, Anhydrate (Fine DCPA) Processing:

DCPA is milled with ˜40 ml alcohol per 100 grams of DCPA until 50% of the resultant particles are below about 2.5 microns in diameter. Typical milling time required at 60 r.p.m. is approximately 3 hours. The alcohol is then removed from the DCPA by drying and the mill media is then removed by sieving.

Two-Step Whiskering Process:

Milled TTCP and Fine DCPA are combined in molar quantities between 1:1 to 1:2 and homogenized then whiskered in a first whiskering solution. The first whiskering solution may include any of the whiskering solution described in the patents and patent applications referenced above. In one non-limiting embodiment, a preferred first whiskering solution may be deionized water chilled to 0°-15° C. The whiskering step is performed at liquid/solid of about 22-44 ml of the first whiskering solution for every gram of combined powders to be whiskered. The powder and liquid are combined with stirring for several minutes (e.g. ˜5 min). The powder is separated from the solution as described earlier, such e.g., by vacuum filtration. The captured powder is then rinsed 1 to 3 times with chilled rinse solutions. In certain cases the rinse solutions may contain 0 to 10 mMol MgCl₂. Typically the final rinse is performed with deionized water without MgCl₂. The excess water is dried off in a drying oven at 50° C. to 110° C.

A second whiskering solution is prepared using about 1 part ortho-phosphoric acid with about 58.65 parts deionized water. The combined powders already whiskered once are whiskered a second time using second whiskering solution at liquid/solid of 0.32 ml per gram powders and dried in an oven at 50° C. to 110° C.

Cement Powder Milling:

The whiskered cement powder is dry milled for approximately 2 minutes to 60 minutes using a mortar and pestle or a ball mill to achieve a particle size distribution such that 50% of the particles are below approximately 3.5 to 6.5 microns and more preferably below 4.4 to 5.2 microns. A portion of the dry milled powder is then milled further such that 50% of the particles are below approximately 3.5 microns and the specific surface area is greater than about 4 m²/g. This can be accomplished in a mechanical mill such as a ball mill using alcohol in the ratio of 0.4 ml alcohol per gram of powder. Typical milling time at 60 r.p.m. is 3.5 hours. A mixture of the two different particle sizes is then blended with calcium oxide in the amount of 0.5% to 1.0% to form the final cement powder mixture. The typical mixture of dry milled and wet milled powders is 15% to 100% dry milled powder by weight. A preferable combination due to desirous handling and setting properties is 30% dry milled and 70% wet milled powders.

Cement Paste Preparation:

The final powder is mixed with the setting liquid using a spatula or equivalent mixing device at a liquid/solid of about 0.27-0.53 (depending on the desired consistency). An example of a preferred setting liquid is 0.4 molar dibasic sodium phosphate with a pH of 9.0. Another example of a preferred setting liquid is a solution of pH 5.6 which contains about 1 part ortho-phosphoric acid (oPA) with 7.35 parts DI water and pH adjusted using sodium hydroxide. Another example of a preferred setting liquid is the above setting liquid adjusted to pH 5.3 using additional oPA. Yet another example of a preferred setting liquid is either of the two described Mixing solutions above with ½ of the moles of sodium replaced with an equal number of moles of potassium using potassium hydroxide. Alternatively, these setting liquid can be made by starting with dibasic sodium phosphate and dibasic potassium phosphate followed by the addition of oPA to attain the desired pH and overall phosphate concentration. Another example of a preferred setting liquid contains any of the above combinations with the addition of up to 10 mM MgCl₂. Another example of a preferred setting liquid is a solution in which some or all of the sodium and potassium ions are replaced with ammonium ions such as by using dibasic ammonium phosphate or ammonium hydroxide in the steps above.

Regardless of the method used to manufacture calcium phosphate particles, a portion of the dissolved calcium may react with dissolved phosphate ions in the aqueous surroundings to form amorphous calcium phosphate precipitate. This precipitate may further contribute to the size and shape of calcium phosphate whiskers.

In an embodiment, whiskered TTCP particles may be contacted with a setting solution and heated to result in a hardened apatitic cement suitable for use as an injectable bone filler material, or for use in the manufacture of prosthetic bone implants.

Modified calcium phosphate cement compositions suited for use in the presently described embodiments may be chosen according certain chemical and/or physical properties that are advantageous for therapeutic use. It is desirable that the constituent CPCs used herein have the ability to harden into cements having high compressive strength. Typically, a CPC composition will be chosen such that a hardened cement made therefrom has a compressive strength of >30 MP, >50 MPa, or >100 MPa. A CPC composition may also be chosen such that, when mixed with an appropriate setting solution, a paste having sufficient viscosity so as to allow the paste to be injected through a syringe or other aperture to a site within a body or a mold will be formed. The preceding two parameters are, at least in part, related to the density of whiskers on the surface of constituent calcium phosphate particles, and to the density of particles comprising the paste. The density of surface whiskers will typically be in a range such that the resulting material has the desired characteristics of being non-dispersive and able to withstand high compressive forces, while allowing the paste to remain injectable. Typically, such characteristics may be realized when the density of surface whiskers is >2.0/μm² and less than 100/μm².

In order for CPC materials to be of therapeutic use in a point-of-care setting, a paste made therefrom should have a setting time and working time that is greater than 1 minute and less than 45 minutes. U.S. Patent Application No. 2005/0069479 to Lin et al. entitled “METHOD OF INCREASING WORKING TIME OF TETRACALCIUM PHOSPHATE CEMENT PASTE,” discloses methods to manipulate the setting and working times of various calcium phosphate compositions. By heating a TTCP paste to between about 50° C. to 350° C. for at least one minute, a paste having a working time and setting time of between about 8 to 45 minutes and about 9.5 minutes to about one hour, respectively, is achieved.

Calcium Phosphate Prosthetic Bone Implants

The prosthetic bone implants suitable for use in the presently described embodiments will be those implants that are made from hardened, bioresorbable calcium phosphate cements (CPC) having apatite as its major phase, without limitation on the structure and/or configuration of the prosthetic bone implants. The apatite comprising the implant body will typically be made without a sintering step. The lack of a sintering step preserves micro- and nano-sized porosity of the calcium phosphate material and allows for improved wicking of body fluids and infiltration of the implant by cells (e.g. osteoblasts, osteoclasts and supportive cells) when compared to implants that are made from conventional sintered CPC.

The hardened CPC will typically be at least partially porous (e.g., as a “porous block”) and may accommodate up to about 90% porosity by volume. In general, interconnected porosity of a calcium phosphate implant is directly related to its bioresorbability, and inversely related to its compressive strength. The relationship between porosity, bioresorbability and compressive strength of an implanted may be exploited to develop an implant having both high compressive strength (typically >50 MPa and up to 170 MPa), and high bioresorbability. In an embodiment, the subject prosthetic bone implants may be adapted to withstand compressive forces equal to or in excess of those typically exerted on naturally occurring bone may be accomplished by coupling hardened calcium phosphate articles having different porosities to each other in configurations that are optimally suited for implantation of the implant in or near a bone of a subject. Typically, a dense CPC block will be less than 40% by volume and will function in a load bearing capacity, whereas a porous CPC block will be 20-90% by volume. The porosity of the calcium phosphate matrix may be controlled by altering one or more process and or composition parameters during manufacture of the implant. By way of non-limiting example, the porosity of an implant may be readily controlled by, for example, including a pore forming powder in the CPC composition, or changing the ratio of pore forming agents in the CPC. In some embodiments, the porosity of the implant may be constant throughout the calcium phosphate matrix.

By way of non-limiting example, prosthetic bone implants well suited to present embodiments are described in U.S. Pat. No. 7,118,705 to Lin et al., entitled “METHOD OF MAKING A MOLDED CALCIUM PHOSPHATE ARTICLE”; U.S. Pat. Nos. 7,119,038; 7,097,793; and 7,083,750 to Lin et al., entitled “METHOD FOR MAKING A POROUS CALCIUM PHOSPHATE ARTICLE”; U.S. Pat. Nos. 6,994,726; 7,115,222; 7,083,749; 7,118,695; and 7,097,792 to Lin et al., entitled “DUAL FUNCTION PROSTHETIC BONE IMPLANT AND METHOD FOR PREPARING SAME.” The above-cited patents and patent applications are commonly owned with the present invention and the contents thereof are hereby incorporated by reference in their entirety as though fully set forth herein. The unsintered prosthetic bone implants described by Lin are biocompatible, bioresorbable, and can be adapted to withstand compressive forces up to 170 MPa.

In some embodiments, an implant may be adapted to have varying porosity throughout the calcium phosphate matrix. The implant may optionally be configured to functionally and structurally mimic the configuration of natural occurring bone, with a denser, load bearing cortical portion, and one or more porous cancellous portions integrally disposed therein. Such a configuration may optimize penetration of body fluids and tissue ingrowth into the implant body. In some embodiments, an implant may have a load bearing cortical portion having at least two opposite surfaces and a cancellous portion integrally disposed in the cortical portion and being exposed through the two opposite sides. Both the cancellous portion and the cortical portion may be formed from hardened calcium phosphate cement. In some embodiments, the cancellous portion may have a porosity that is greater than the porosity of the cortical portion. The porosity of the cancellous portion may be at least about 20% by volume. In some embodiments, the cortical portion may also be formed from a porous calcium phosphate cement. The cortical portion may have a porosity of less than about 40% by volume.

In addition to the implant configurations disclosed in the above-cited references, certain embodiments may be directed to implants having improved bioresorbability properties. Improved bioresorbability may be realized, at least in part, by including an additional layer of nano- and micro-sized porosity to the surface of the implant. In embodiments, the outer porous layer will be at least 100 μm in thickness. Implants that incorporate such an outer porous layer will exhibit improved wicking profiles, and may allow body fluids, vascularization and cellular infiltration of the implant from the exterior of the implant. Such may be readily achieved by coupling a porous component to the exterior surface of the implant during the manufacture thereof. Alternatively, an exterior porous layer may be formed on the surface of the implant by subjecting the implant surface to a treatment that forms a layer of porous calcium phosphate material on the surface thereof. Such treatments are described in detail below.

The bioresorbability of a calcium phosphate implant is related the size and interconnectedness of pores distributed throughout the matrix or body of the implant. Ideally, the pores will be large enough to allow body fluid wicking and osteoblast infiltration. Typically, infiltration of osteoblasts is facilitated when at least a portion of the pores have openings and/or pore throat diameters of approximately 100 μm or larger. Pore throat diameter and pore opening diameter ranges may be from 100-500 μm to about 100-300 μm, respectively.

In an embodiment, pore size and pore throat diameters may be manipulated by selecting a pore forming powder having an average particle size in the desired pore size range. Moreover, incorporating salts having varying crystal geometry into the cement paste may improve the interconnected porosity of an implant. In an embodiment, the relative degree of interconnected porosity of an implant may be manipulated by varying the ratio of pore forming powders having different size and geometry (e.g. combination of spherical and cuboidal crystals). A non-limiting example of a salt having a spherical crystal structure is KCl. A non-limiting example of a salt having cuboidal crystal structure is NaCl. Generally, spherical salt particles will have less adverse effect on the mechanical strength of the implant, but do not allow maximum interconnected porosity. The lack of interconnected communication between adjacent pores in the implant body may be remedied by including non-spherical salt crystals therein. The degree of interconnected porosity may be further manipulated by varying the ratio of spherical to non-spherical salt crystals. In an embodiment, the ratio of spherical to non-spherical salt crystals comprising the pore forming powder will be from about 9:1 to about 1:4, or from 3:4 to about 1:4. In an embodiment, the ratio of spherical to non-spherical salt crystals comprising the pore forming powder may be about 1:1.

In an embodiment, interconnected porosity and pore size may be influenced by the average particle size of constituent particles comprising the hardened cement. Typically, CPC particles having an average diameter between about 0.1 μm to about 500 μm are used to form the implants.

Typically, when forming CaP implants having interconnected porosity, the ratio of pore-forming powder to CPC powder (dry weight ratio) will not exceed about 1:1. Using higher ratios may adversely affect the compressive strength of the resulting implant.

In a further non-limiting embodiment, microcavities and or internal voids may be created in the body of the subject implants by suspending salt crystals. The density of particles is such that they do not substantially touch adjacent particles. The particles may function as drug reservoirs when the drug is loaded therein. Advantageously, the microcavities formed in this manner may serve as reservoirs for bioactive compositions, thus increasing the elution time and or effective treatment time of a pharmaceutical agent.

The pore forming powder may be removed from the hardened calcium phosphate implant by soaking the implant in an aqueous solution, as set forth in above-cited references.

A bone implant, as described herein, may be used to replace a portion of a human bone or bone system. For example, bone implants may be used to replace disks in a human spine. In some embodiments disk replacement in the C5-C7 region of the spine may be performed using the bone implants disclosed herein. A bone implant may be at least partially bioresorbable over time. In an embodiment, a bone implant may be at least partially composed of calcium phosphate to enhance the bioresorption of the implant.

The bone implant may have a shape that allows the implant to match the bone that the implant is used to replace. A bone implant may have a circular, oval, elongated disk, ring, square, rectangular, or irregular cross-sectional shape. In other embodiments, a bone implant may be U-shaped, C-shaped, an elongated ring with a gap, a disk with an orifice, or an elongated disk with an orifice. A bone implant may have a shape similar to a disk in a spine. For use in spinal applications, a bone implant may have a length of about 1 cm to 5 cm or about 2 cm to about 2.5 cm and a height of about 1 mm to 20 mm or about 2 mm to about 15 mm.

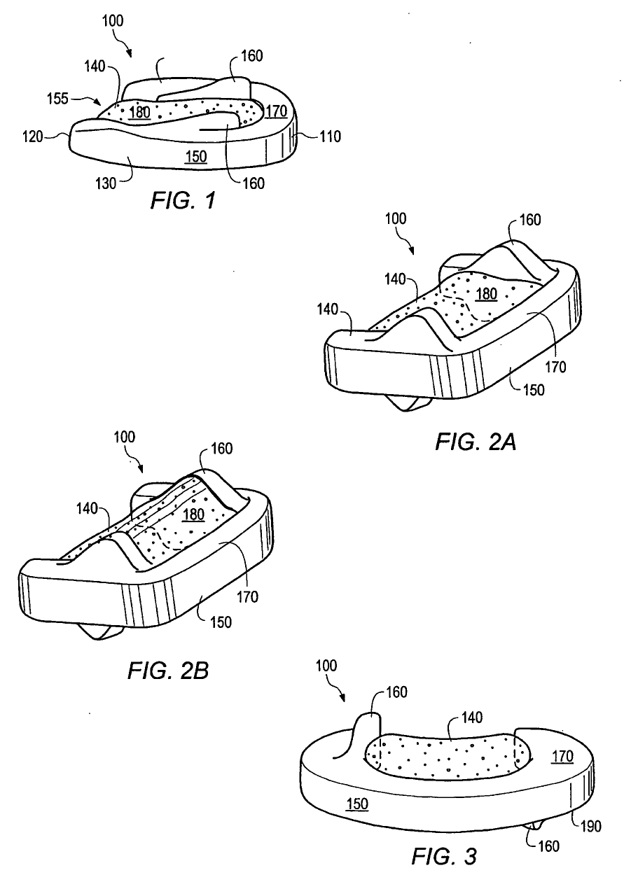

An embodiment of a bone implant suitable for use in a spinal disk replacement process is depicted in FIG. 1. As seen in FIG. 1 implant 100 may be tapered from a first end 110 to a second end 120. Tapering an implant may facilitate maintenance of a natural lordosis of a spine.

Implant 100 may include one or more porous components 140, a load bearing component 150, and one or more protrusions 160. A porous component, in the context of this application, is a component with a greater porosity than the load bearing component. A porous component and/or a load bearing component may be composed of a hardened calcium phosphate as previously described. In an embodiment, a porous component may have a cross-sectional shape similar to the bone implant. Porous component(s) 140 may have a circular, oval, elongated disk, ring, square, rectangular, U-shaped and/or irregular cross-sectional shape.

One or more openings may extend into and/or through one or more of the porous components. Openings in the porous component may be positioned at an approximate center or clustered around an approximate center of a porous component. In an embodiment, an opening may be positioned away from an interface of a porous component and the load bearing component. It is believed that openings proximate an interface between a porous component and a load bearing component may weaken the interface. In some embodiments, additives such as bone marrow, blood, blood cells, or bone growth promoting material may be placed within one or more openings. One or more openings may be configured to be able to receive and at least partially retain additives.

As stated previously, a bone implant may be bioresorbable over time. Use of a porous component may encourage callus growth, thus improving the bioresorbability of the implant. Bioresorption of an implant used as a spinal disk replacement implant may promote bone fusion of adjoining vertebrae.

Turning back to FIG. 1, load bearing component 150 at least partially surrounds porous component 160. In some embodiments, load bearing component 150 completely surrounds a porous component portion of porous component 160. In other embodiments, load bearing component 150 is C- or U-shaped. In such embodiments, a portion of the porous component is not surrounded by the implant, as depicted in FIGS. 1-6. As depicted in FIG. 1, load bearing component may leave a gap 155 where the load bearing component does not surround porous component 140.

A load bearing component may have greater compressive and/or shear strength than a porous component. A bone implant may be designed so that the compressive forces on the bone implant are transferred to the load bearing component during use. A load bearing component may have a substantially circular, oval, elongated ring, ring, square, rectangular, or irregular cross-sectional shape. In other embodiments, the load bearing component may be U-shaped, C-shaped, an elongated ring with a gap, a disk with an orifice, or an elongated disk with an orifice.

The load bearing component may have a similar or dissimilar cross-sectional shape to one or more of the porous components. In some embodiments, a load bearing component may have a length of about 1 cm to 5 cm or about 2 cm to about 2.5 cm and a height of about 1 mm to 20 mm or about 2 mm to about 15 mm

A load bearing component may include one or more protrusions. One or more protrusions may retain a bone implant in a desired position when implanted in a body. In embodiments when the bone implant is a spinal implant, one or more protrusions may retain a tapered implant within a spine to maintain a natural lordosis. In spinal applications, a protrusion of a bone implant may engage an endplate of a vertebra. Penetration of a protrusion into an endplate of a vertebra may be enhanced if the protrusion is tapered away from a surface on which the protrusion is positioned. In some embodiments, a protrusion may be positioned in an opening created in a bone during implantation of the implant. Implant protrusions may reduce and/or eliminate the need for the use of a cervical plate during a disk replacement spinal surgery.

A protrusion may be a coupled to load bearing component 150, as depicted in FIG. 1. In some embodiments, protrusion 160 may be attached to a load bearing component. In other embodiments, protrusion 160 is formed from a portion of the load bearing component. Protrusions 160 may extend beyond a top surface 170 of a load bearing component 150 and/or a bottom surface of the load bearing component, see FIGS. 1 and 2A. A protrusion may be about 1 to about 5 mm high and less than about 2.5 cm wide.

A protrusion may have a substantially triangular, U-shaped, arch shaped, or irregular cross-sectional shape. A protrusion may have a similar shape to a load bearing component. For example, a load bearing component with a U-shaped cross-sectional shape when viewed from a surface, may have a U-shaped protrusion on the surface that follows the shape of the load bearing component. A protrusion may taper away from a surface of the load bearing component. For example, as shown in FIG. 1, a protrusion 160 may taper from a first end 110 to a second end 120.

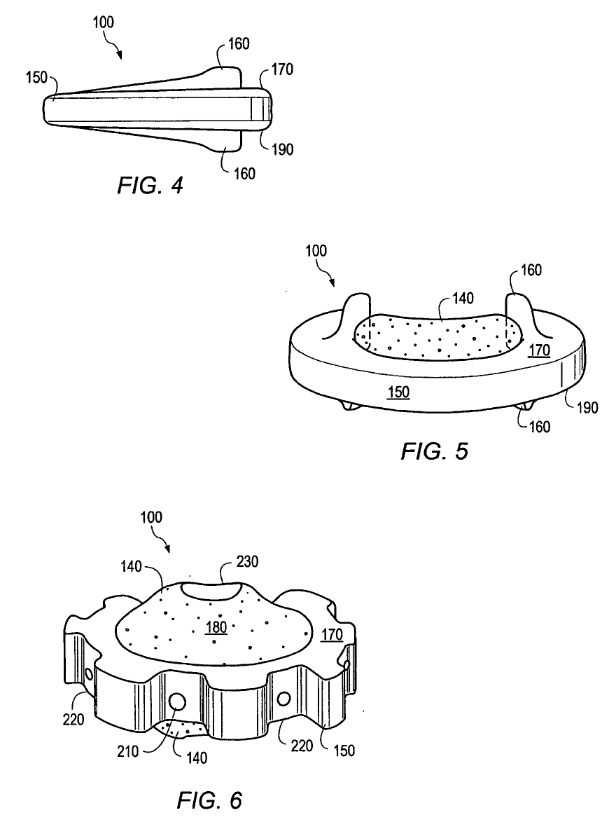

A protrusion 160 may be positioned on a surface 170 of a load bearing component 150, as shown in FIG. 1. In an embodiment, a protrusion 160 may be positioned on a first surface 170 and a protrusion may be positioned on a second surface 190 of a load bearing component 150 of an implant 100 diametrically to each other, see FIG. 3. Alternatively, a protrusion 160 may be positioned on a first surface 170 and a protrusion may be positioned on a second surface 190 such that the protrusions are substantially mirrored on each side, see FIG. 4. Two or more protrusions on a bone implant may be positioned on each side of an implant, see FIGS. 2A, 2V, and 5. In an embodiment, an implant 100 may include four protrusions 160 on a load bearing component that extend beyond a surface of a load bearing component 150 and a porous component 140, see FIG. 5. Protrusions may be positioned on or near an edge of a load bearing component.

A load bearing component 150 of a bone implant 100 may include one or more apertures 210, see FIG. 6. An aperture may be an opening that extends through a load bearing component. Apertures formed in the load bearing component may promote callus growth through the aperture and, therefore, through the implant. Additives may be incorporated into the implant by inserting additives into one or more of the apertures. Additives that may be inserted include porous components, blood, blood cells, bone marrow, and other materials that enhance bone growth around and through the implant. Apertures may extend through any surface of the load bearing component. In an embodiment, apertures may be positioned on a curved end of load bearing component.

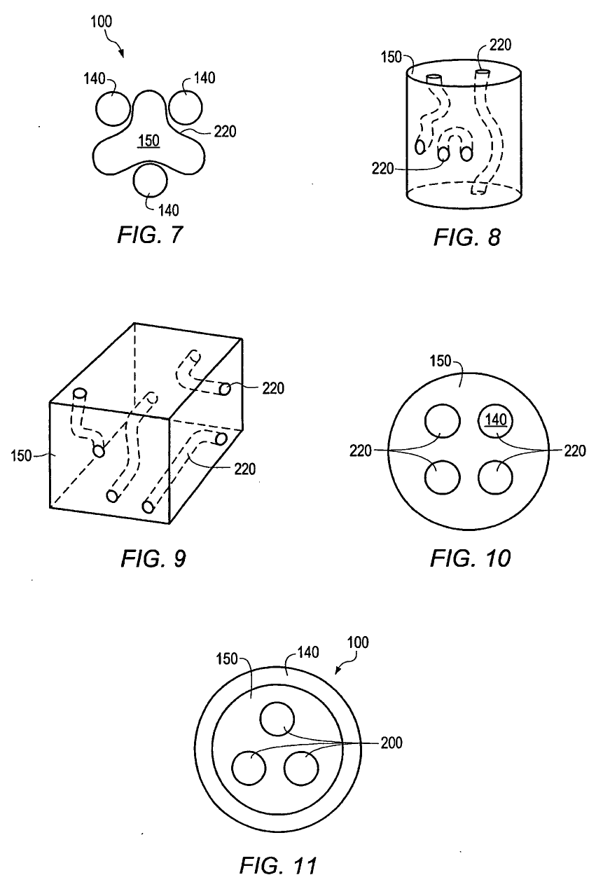

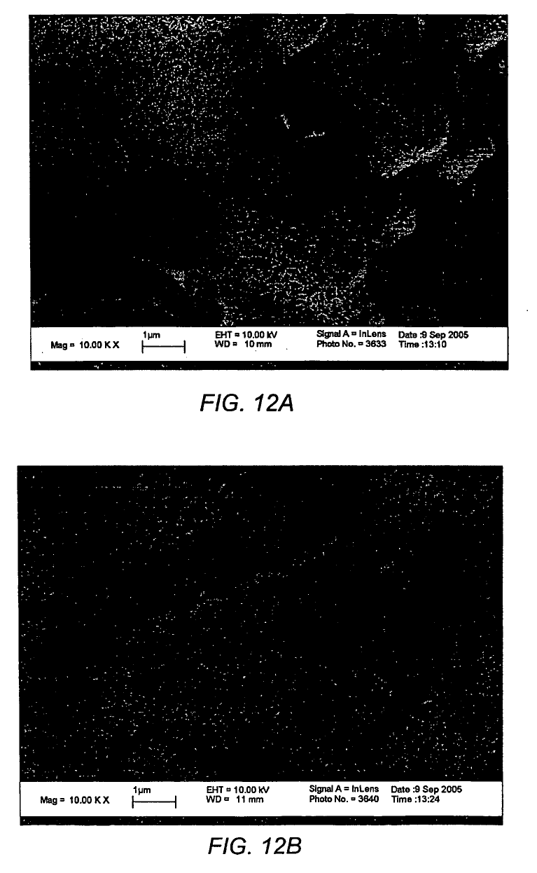

In some embodiments, a bone implant 100 may include a load bearing component 150 with one or more channels 220, see FIGS. 6 and 7. Channels may be on an exterior surface of the load bearing component and/or an interior of the load bearing component. Channels positioned on an exterior of the load bearing component may promote callus growth on an exterior surface of the load bearing component. A bone implant with a load bearing component with several channels may have a shape similar to a gear with a gap, see FIG. 6. Callus may form in the channels of the gear shaped bone implant.

In an embodiment, a surface 180 of a porous component 140 may extend beyond a surface 170 of a load bearing component. A surgeon may create a groove in a vertebra to receive the portion of the porous component that extends beyond a surface of a load bearing component. Extending a surface of a porous component beyond a surface of a load bearing component may help retain the bone implant in a desired position in a spine and help promote callus growth between adjacent vertebrae. In an embodiment, two opposing surfaces of a porous component may extend beyond proximate surfaces of the load bearing component. An opening 230 in a surface 180 of the porous component 140 may extend through the porous component.

In some embodiments, channels 220 may extend throughout a load bearing component 150, see FIGS. 8-10. A bone implant with several channels extending throughout a load bearing component may have structure similar to a sponge where channels may wind through a load bearing component from one surface to another surface. Channels may be sized so that the strength of the load bearing component is not compromised by the channels, i.e., the load bearing component still has a strength that is large enough to withstand compressive forces on the implant. Using several channels throughout an interior of a load bearing component may promote bone fusion and/or callus growth through the channels. Porous components 140 and/or other additives may be positioned in the channels in the load bearing components to further promote implant resorption. In an embodiment, a bone implant 100 may include a load bearing component 150 with several channels 220 and a surface of porous component 140, see FIG. 11. A surface of porous component on an exterior of a load bearing component may promote the growth of callus, during use in a spine.

In some embodiments, an implant may be formed from calcium phosphate cements using molds. Formation of molded calcium phosphate implants is described in U.S. Pat. No. 6,994,726 to Lin et al., entitled “DUAL FUNCTION PROSTHETIC BONE IMPLANT AND METHOD FOR PREPARING SAME.”

A bone implant may optionally include one or more mesh restrictors placed in association with an outer surface or an outer edge of the implant. The mesh restrictor may be wrapped around the outer edge of the implant. Alternatively, a mesh restrictor may be placed into the material that will form the implant body, during its manufacture and prior to the hardening of the CPC paste that will form the implant body. A restrictor may be made of any non-toxic, malleable material, such as a thermoplastic material. Mesh restrictors may be woven, wrapped, or formed as a sheet.

Bioactive Compositions

In some embodiments, incorporating one or more bioactive agents into a prosthetic implant may enhance the biocompatibility and/or bioresorbability of the implant. Constituents of the bioactive composition may be selected to impart certain advantageous therapeutic or physiological properties on the implant.

In an embodiment, bioactive agents may include one or more osteoinductive compounds. The local inclusion of one or more osteoinductive compounds with the implant in situ may accelerate healing, vascularization, tissue and cellular infiltration of the implant. Suitable osteoinductive compounds include osteogenic compounds. Numerous osteogenic compounds are known to practitioners of ordinary skill in the art including any one of a number of polypeptide growth factors known for their ability to induce the formation or remodeling of bone. By way of non-limiting example, osteogenic compounds suitable for use with the presently described embodiments may include, but are not limited to, osteogenin, Insulin-like Growth Factor (IGF)-1, Transforming Growth Factor (TGF)-β1, TGF-β2, TGF-β3, TGF-β4, TGF-β5, osteoinductive factor (OIF), basic Fibroblast Growth Factor (bFGF), acidic Fibroblast Growth Factor (aFGF), Platelet-Derived Growth Factor (PDGF), vascular endothelial growth factor (VEGF), Growth Hormone (GH), and osteogenic protein-1 (OP-1). In certain embodiments, growth factors belonging to the Bone Morphogenic Protein (BMP) family of growth factors, which include, but are not limited to, BMP-1, BMP-2A, BMP-2B, BMP-3, BMP-3b, BMP-4, BMP-5, BMP-6, BMP-7, BMP-8, BMP-8b, BMP-9, BMP-10, BMP-11, BMP-12, BMP-13, BMP-14, BMP-15, or combinations thereof, may be especially suited for use with the subject implants.

In some embodiments, bioactive agents may include one or more compounds that support the formation, development and growth of new bone, and/or the remodeling thereof. Typical non-examples of compounds that function in such a supportive capacity include certain bone matrix proteins (e.g., alkaline phosphatase, osteocalcin, bone sialoprotein (BSP) and osteocalcin in secreted phosphoprotein (SPP)-1, type I collagen, type IV collagen, fibronectin, osteonectin, thrombospondin, matrix-gla-protein, SPARC, alkaline phosphatase and osteopontin). In an embodiment, a peptide or peptide fragment may contain the amino acid sequence Arg-Gly-Ser, which has been shown to bind to and enhance the recruitment of osteoblasts.

Bioactive agents may, in some embodiments, further include pharmacologically active compounds that do not act locally to stimulate bone growth and healing, but that nonetheless may confer a therapeutic advantage in certain applications, such as, for example, antibiotic and or analgesic agents. Exemplary analgesic agents suitable for use herein include, but are not limited to, norepinephrine, bupivacaine, ropivacaine, 2-chloroprocaine, lidocaine, mepivacaine, ropivacaine, mepivacaine, benzocaine, tetracaine, dibucaine, cocaine, prilocaine, dibucaine, procaine, chloroprocaine, prilocaine, mepivacaine, etidocaine, tetracaine, xylocaine, morphine, fentanyl, alphaxalone and active analogs, 5-alpha-pregnane-3 alpha-21-diol-20-one (tetrahydro-deoxycorticosterone or THDOC), allotetrahydrocortisone, dehydroepiandrosterone, benzodiapenes, nifedipine, nitrendipine, verapamil, aminopyridine, benzamil, diazoxide, 5,5 diphenylhydantoin, minoxidil, tetrethylammonium, valproic acid, aminopyrine, phenazone, dipyrone, apazone, phenylbutazone, clonidine, taxol, colchicines, vincristine, vinblastine, levorphanol, racemorphan, levallorphan, dextromethorphan, cyclorphan, butorphanol, codeine, heterocodeine, morphinone, dihydromorphine, dihydrocodeine, dihydromorphinone, dihydrocodeinone, 6-desoxymorphine, heroin, oxymorphone, oxycodone, 6-methylene-dihydromorphine, hydrocodone, hydromorphone, metopon, apomorphine, normorphine, N-(2-phenylethyl)-normorphine, etorphine, buprenorphine, phenazocine, pentazocine and cyclazocine, meperidine, diphenoxylate, ketobemidone, anileridine, piminodine, fentanil, ethoheptazine, alphaprodine, betaprodine, 1-methyl-4-phenyl-1,2,5,6-tetrahydropyridine (MPTP), loperamide, sufentanil, alfentanil, remifentanil, lofentanil, 6,7-benzomorphans, ketazocine, aryl-acetamides, U-50,488, spiradoline (U-62,066), enadoline (CI-977), asimadoline, EMD-61753, naltrexone, naltrindole.

Exemplary though non-limiting antibiotic agents include, but are not limited to, tylosin tartrate, tylosin, oxytetracycline, tilmicosin phosphate, ceftiofur hydrochloride, ceftiofur sodium, sulfadimethoxine cefamandole, tobramycin, penicillin, cefoxitin, oxacillin, vancomycin, cephalosporin C, cephalexin, cefaclor, cefamandole, ciprofloxacin, bisphosphonates, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericine B, ketoconazole, fluconazole, pyrimethamine, sulfadiazine, clindamycin, lincomycin, pentamidine, atovaquone, paromomycin, diclarazaril, acyclovir, trifluorouridine, foscarnet, penicillin, gentamicin, ganciclovir, iatroconazole, miconazole, Zn-pyrithione.

The amount of a pharmacologically active agent to include in the subject bioactive coating compositions may typically vary with the identity of the agent, the physiological context in which the agent is being employed, and the magnitude of the desired response. Typical dosages of pharmacologically active agents that will be loaded onto the calcium phosphate carrier may be in the range of 2 ng/m³ to 1 mg/m³, according to the volume of pharmaceutical carrier used to deliver the bioactive agent. General guidance in determining effective dose ranges for pharmacologically active compounds may be found, for example, in the publications of the International Conference on Harmonisation and in REMINGTON'S PHARMACEUTICAL SCIENCES, chapters 27 and 28, pp. 484-528 (Mack Publishing Company 1990), which is incorporated by reference as though fully set forth herein.

Bioactive agents may be coupled to the implant by way of a pharmaceutically acceptable carrier. Desirable characteristics for pharmaceutical carriers employed in the presently described embodiments include at least one of i) biocompatibility; ii) bioresorbability; iii) ability of the carrier to stably store the bioactive agents and/or allow its sustained release to surrounding tissues and cells. Such characteristics may be realized using a thin (10-50 μm in thickness) crystalline hydroxyapatite layer formed on the surface of the implant.

Co-Precipitating a Bioactive Composition with Hydroxyapatite

In a first set of embodiments, a method is provided whereby a layer of crystalline calcium phosphate is formed on the surface a calcium phosphate prosthetic bone implant by co-precipitating apatite and one or more bioactive agents from a physiologically acceptable aqueous calcium phosphate solution. The co-precipitated bioactive agents will be associated with the matrix of said crystalline calcium phosphate surface layer. When implanted into recipient bone, bioactive agents are gradually released from the crystalline calcium phosphate layers of the subject implants in a sustained manner. Thus, it is an object of the presently described embodiments to provide an improved prosthetic bone implant comprising unsintered calcium phosphate, that is bioresorbable, biocompatible, and acts as a carrier for therapeutically effective bioactive agents. It is a further object of the present invention to provide methods for the manufacture of such implants.

Calcium phosphate layers produced using current art-recognized techniques are typically composed of large, partially molten HAp particles. HAp produced synthetically under these conditions is prone to delamination and is poorly degraded in situ. The calcium phosphate layers of the present embodiments, in addition to being bioresorbable and biocompatible, are produced under physiological conditions and thus have the additional advantage of being able to integrally accommodate bioactive molecules, such as osteogenic agents, that typically cannot withstand harsh processing treatments (e.g., elevated temperature pressure, osmotic conditions and pH). The bioactive molecules may be co-precipitated with the inorganic mineral components that will form the crystalline calcium phosphate. As a consequence, the bioactive agents are incorporated into the crystal structure of the precipitated mineral coating, rather than being merely deposited upon the surface of the implant and or the coating. In forming an integral part of the calcium phosphate coatings, the elution profile of the integrated bioactive agent is more constant and sustained rather than being a single burst (as when superficially adsorbed). The reduced elution rate advantageously prolongs the osteoinductive and healing potential of therapeutics agents acting locally at the implantation site.

In an embodiment, the crystalline coating may involve the nucleation and growth of HAp crystals on the surface of a calcium phosphate prosthetic bone implant. Unlike similar treatments in prior art coating procedures, the subject implants, being substantially composed of HAp, do not require a pre-treatment process to deposit a nucleating layer on the implant surface, although such a pre-treatment step may be performed if desired.

In an embodiment, formation of the crystalline coating may include contacting the implant with a coating composition that includes a source of calcium and a source of phosphate. Contacting the implant with the coating composition may include fully or partially immersing the implant in the coating composition. Typically, this step will be carried out at a temperature that is within physiologic range (e.g., between about 20° C. to about 45° C., between about 25° C. to about 37° C., or at about 37° C.). The implant will be contacted with the coating composition for a period of time sufficient to allow the precipitation of crystalline calcium phosphate on the surface of the implant. Typically, a layer crystalline calcium phosphate mineral that is at least 0.5 to about 100 μm thick, between 20 to about 50 μm thick, or about 40 μm thick, will be allowed to form on the surface of the implant. Layers of such thickness will typically be achieved in less than 100 hours at 37° C., or more typically, in less than about 48 hours at 37° C. The thickness of a calcium phosphate mineral layer may be monitored using techniques widely familiar to practitioners, such as densitometry, reflectometry, scanning electron microscopy, spectroscopy, or the like.

The coating composition will contain amounts of calcium and phosphate that are sufficient to precipitate crystalline HAp at physiological temperature and pH. The concentration of calcium ions in the coating composition may range from 0.5 to 10 mM, or from 0.5 to 5 mM. The concentration of phosphate ions in the coating composition may range from 0.5 to 6 mM, or from 0.5 to 3 mM. Sodium chloride, or any suitable salt may be added to maintain the ionic strength of the coating composition. Typically the ionic strength of the solution should be between 100 mM to 200 mM sodium chloride, and more typically 150 mM.

The size of HAp crystals may be controlled by varying the amount of crystal growth inhibitors in the coating composition (e.g., magnesium and carbonate), with crystal size being inversely proportion to the concentration of crystal growth inhibitors present in the solution. In order to form HAp crystals, the concentration of magnesium should be less than 7.5 mM, more typically less than 2.5 mM, and most typically less than 0.5 mM. Similarly, HAp crystals ideally form when the concentration of carbonate ions is less than 25 mM, more typically less than 10 mM, and most typically less than 5 mM.

Typically, precipitation of HAp crystals will occur at a substantially physiological pH (from 6-8, or about 7.4). An appropriate buffer, like tris (amino-ethane) or HEPES (N-[2-hydroxyethyl]piperazine-N′-[4-ethanesulfonic acid]) is preferably used to maintain the desired pH. Suitable buffers to maintain a desired pH are known from the art. The relationship between temperature, pH and calcium phosphate solubility per se is known in the art. The skilled practitioner will be able to derive suitable conditions from the guidelines described above. Information and further guidance on solubility calculations for various calcium phosphates may also be found in “G. Vereecke & J. Lemaitre: Calculation of the solubility diagrams in the system Ca(OH)₂—H₃PO₄—KOH—HNO₃—CO₂—H₂O, J. Crystal growth 104 (1990) 820-832.

Generally, the bioactive agents that are to be co-precipitated with HAp crystals will be solubilized in the coating composition. Typically the concentration of the one or more bioactive agents in the solution will be in a concentration range of 0.1 mg/l to 10 g/l, in the range of 0.1-1000 mg/l, in the range of 0.1-500 mg/l, or in the range of 0.1-20 mg/l. Depending upon the desired type of crystals to be grown, the skilled professional may choose to use particular concentrations, pH ranges and temperatures to form the crystals. Most preferably calcium and phosphate are among the inorganic ions used to incorporate bioactive agents into an implant. A coating composition for depositing crystalline HAp on the surface of a calcium phosphate implant will typically be buffered at a pH in the range of 6 to 8.

The pH of the coating composition may depend upon the isoelectric point (pI) of a bioactive agent that is to be incorporated into the coating. Co-precipitation of bioactive agent with inorganic crystals is related to electrostatic interactions. For chargeable compounds, and in particular for amphoteric compounds, the efficiency of incorporation depends on the pI of the bioactive agent and pH of the coating composition. The pI of a compound can be measured by isoelectric focusing polyacrylamide gel electrophoresis. In some embodiments, the bioactive agent is charged at the pH at which the bioactive agent is incorporated into the implant, because this positively affects the amount of bioactive agent that is incorporated.

For the purpose of non-limiting illustration, BMP-2 has a IEP of 9.2. Accordingly the protein has a positive charge below 9.2 and negative charge above 9.2. At a pH of 7.4 for the coating composition, the protein is positively charged and thereby interacts with anions (such as phosphate) in solution. The interaction of the protein with the anions, enhances co-precipitation thereof with HAp crystals growing on the implant surface. For instance, a concentration of BMP-2 in a coating composition of 5 mg/L may lead to an incorporation of 5 μg/mg of coating at pH 7.4. BMP-7, however, has an IEP of 7.7. At a pH of 7.4, the efficiency for incorporation is low due to insufficient difference between IEP and coating pH. Under the same conditions, the incorporation of BMP-7 is only 0.25 μg/mg coating at pH 7.4 for 5 mg/l of BMP-7 in coating solution. In order to increase efficiency of incorporation, a lower pH for coating solution may be selected (e.g. 6.7). Ideally, the difference between pH and pI for each bioactive agent in the composition should be at least about 1 pH unit for optimal co-precipitation of bioactive agents with the growing inorganic layer. For basic amphoteric compounds (pI>7.0) co-precipitation is preferably performed at a pH below pI, for acidic amphoteric compound (pI<7.0) co-precipitation is preferably performed at a pH higher than pI. For compounds with a pI of 7.0 a pH close to 6 or close to 8 is preferred. In case several compounds with different pI's are to be incorporated, it is preferred to choose a pH where all bioactive agents are charged, if possible. If this is not possible, more than one co-precipitation procedure may be performed, with each procedure incorporating bioactive compositions using conditions are close to ideal as possible, resulting in an implant with more than one crystalline coating. This may be advantageous in some cases, since in vivo, HAp crystals typically degrade from the outside in. Thus, therapeutic agent such as osteogenic compounds and analgesic compounds may be precipitated on an outer layer of the implant, while therapeutic agents such a bone proteins or antibiotics may be deposited first.

The pH of the calcium phosphate solution typically has less influence on the incorporation rate of uncharged bioactive agents. In general physiological pH, around 7.4 is suitable for this purpose.

In an embodiment, including one or more bioactive agents, in particular one or more osteoinductive agents, in the coating may stimulate cell activity and cell differentiation near an implant. Accordingly, the subject coated implants may regenerate or repair bone tissue more efficiently and more rapidly than implants which do not contain bioactive agents. The release of bioactive agent(s) is related to the rate of coating degradation. After implantation, the mineral coating is remodeled or degraded by osteoclastic activity, leading to a gradual release of the bioactive agent(s), around the implanted medical device. Thus an optimal concentration of bioactive agent(s) can be maintained around the medical device, and burst-release of bioactive agent(s), which may lead to unwanted side effects and premature cessation of therapeutic activity of the implant may be avoided.

In vitro, the degradation of the coating and release of the bioactive agent(s) may be monitored by measuring the calcium and or bioactive agent(s) release under physiological conditions as a function of time. Methods to monitor levels of these compounds are known in the art and include monitoring via a calcium-ion selective electrode, chromatography or enzyme-linked immunosorbant assay to measure the elution profiles of polypeptide factors. Ideally, a growth factor incorporated into a crystalline calcium phosphate layer as described herein will have an elution profile at physiological pH (about 7.4) that roughly corresponds to the dissolution rate of the calcium phosphate matrix in which it is incorporated.

Optionally, it may be desirable, under certain situations to “pre-coat” the surface of the prosthetic bone implant with an initial layer (e.g., an amorphous mineral) of inorganic compounds, such as with an initial layer comprising calcium and phosphate. The amorphous layer may be obtained by contacting the implant surface with an aqueous calcium phosphate pre-coat solution under high nucleation conditions to obtain a thin and amorphous calcium phosphate layer. The optional amorphous layer may act as a seed to enhance the ability of more highly structured crystalline HAp to be precipitated on the implant surface. In some applications, including the optional amorphous layer may improve the stability and the activity of the crystalline HAp coating and the bioactive agent(s) incorporated therein. The implant may be pre-coated for a period of time sufficient to deposit an amorphous layer of calcium phosphate material at least 1 μm in thickness (typically, between 12-24 hours).

The composition of the inorganic components of the pre-coat solution may be chemically similar to that found in body fluids. The concentration of calcium ions in the pre-coat solution may range from 0.5 to 20 mM, or from 8 to 12.5 mM. The concentration of phosphate in the pre-coat solution may range from 0.5 to 10 mM, or from 2.5 to 5 mM. The concentrations of calcium and phosphate may have to be adjusted to maintain a desired pH. The solubility of calcium phosphate is inversely proportional to pH, that is, as pH increases the solubility of calcium phosphate decreases. For example, at 37° C., and at a pH of 6.7, calcium phosphate is more soluble than at physiological pH (about 7.4). Concentrations of calcium and phosphate, in some embodiments, will be between 4 mM to 15 mM for calcium and 2 mM to 20 mM for phosphate.

Furthermore, the presence of magnesium ions is thought to inhibit the deposition of crystalline calcium phosphate mineral coatings. Particularly, the presence of magnesium has been found to inhibit or reduce the crystal growth of the coating during deposition from the calcium phosphate solution, resulting in an amorphous calcium phosphate layer that may act as a seed to enhance formation of crystalline HAp subsequently precipitated thereon. Optimum control of crystal growth leads to a uniform, strong and wear resistant coating. Magnesium and carbonate ions may be present in the pre-coat solution at concentrations below 10 and 25 mM, respectively. The quantity of magnesium and carbonate, both inhibitors of crystal growth may be adjusted for optimal formation and attachment of the optional amorphous pre-coat layer. In embodiments where apatite crystals are to be formed it is desirable to produce apatite crystals of submicrometer dimensions (<1 microns), which may result in a mechanically stronger coating. The average size of the crystals may be decreased by increasing the magnesium and carbonate ion concentration.

Formation of Nanoporous Nanocrystalline HAp

In an alternate embodiment, bioactive compositions may be coupled to prosthetic bone implants by first forming a layer of nanoporous HAp nanocrystals on the surface of at least a portion of the implant. Nanoporous HAp nanocrystals may also be formed on the surface of a calcium phosphate implant surfaces using any art-recognized technique. In some embodiments, the nanocrystalline HAp surface will be highly porous and have a surface area in the range of about 25 m²/g to about 150 m²/g. The surface area of the nanocrystalline HAp coating the subject implants will be directly proportional to amount of bioactive composition that can be coupled to the implant. Advantageously, the surface area of the nanocrystalline HAp coating is inversely related to the elution rate of the bioactive composition when implanted in a subject.

FIG. 1 shows SEM images (at 10,000 fold magnification) of the surface of calcium phosphate implants having a surface layer of nanoporous HAp nanocrystals according to some embodiments. Individual particles of calcium phosphate are cemented to each other, and a layer of nanoporous HAp nanocrystals is formed thereon.

In one non-limiting embodiment, a layer of nanoporous HAp nanocrystals that is well suited for prolonged retention and slow elution of bioactive agents (such as drugs, growth factors or other agents having biological activity) may be formed on the surface of a CaP implant by contacting the portion of the implant that is to be coated with an aqueous solution containing a source of phosphate ions. Optionally, the solution may contain a source of calcium ions. The implant will be soaked in the solution for a period of time that is sufficient to form nanocrystalline HAp on the implant surface. In an embodiment, the implant may be soaked for up to 8 days. After soaking, the implant may be rinsed with the solution, with water, or with an appropriate physiological buffer. Optionally, the implant may be dried and stored under sterile conditions for use in a point-of-care setting.

In an embodiment, the nanoporous nanocrystalline HAp layer will have a surface area of between about 25 m²/g to about 150 m²/g, or between about 60 m²/g to about 100 m²/g. The increased surface area of the prosthetic bone implants significantly increases the drug binding capacity of the implant (i.e. results in a greater amount of bioactive composition to be coupled thereto).

In an embodiment, the physical and chemical properties of surface nanoporous HAp nanocrystals may be by altered by including one or more additives in the aqueous solution. The additives may include, for example, inhibitors of crystal formation, such as magnesium and/or carbonate ions (as described extensively above). By controlling the amount of such additives in an aqueous solution, the morphology of nanoporous HAp nanocrystals may be regulated.

In an embodiment, the physical and chemical properties of surface nanoporous HAp nanocrystals may also be determined by the amount of time that the implant is left in contact with the aqueous solution. Typically, the implant will be contacted with the aqueous solution for a period of time ranging from between 1 to 8 days. The amount of time that the implant is to be contacted with the aqueous solution is dependent on factors such as the chemical composition of the solution, and the surface area that is desired. FIG. 1 demonstrates the dependence of the surface area nanocrystalline HAp on chemical composition and incubation time. FIGS. 1A and 1B each show an SEM image of 10,000-fold magnification of the surface of a hardened calcium phosphate cement that has undergone the indicated treatment. The image depicted in FIG. 1A corresponds to a CaP sample that has been immersed in Hank's Balanced Salt Solution (HBSS, with calcium and magnesium) for 3 days. The image depicted in FIG. 1B corresponds to a CaP sample that has been immersed in Phosphate Buffered Saline (PBS) for 5 days. Nanophase nanocrystalline HAp is formed under both sets of conditions.

Alternatively, deposition of nanophase HAp nanocrystals on the surface of the subject implants may be performed using techniques such as ion-spray or sol-gel surface chemistry techniques. Formation of nanophase HAp nanocrystals typically occurs under physiologically unfavorable conditions and may be performed in the absence of bioactive agents whose stabilities are intolerant to such conditions. In these cases, the implant and nanophase HAp coating may be prepared and packaged under ascetic conditions. Bioactive agents may be loaded onto the surface thereof in a point-of-care setting by immersing the coated prosthetic bone implant in a sterile, physiologically buffered aqueous solution containing the dissolved bioactive composition. After loading onto the implant, the implant is delivered to its desired site in the body. Due to its high surface area and affinity for polypeptides, in particular BMPs, the elution rate of bioactive agents from the nanophase HAp is similar to the dissolution rate of the HAp crystals.

Bioactive compositions may be loaded onto the CaP subject bone implant by soaking the implant in an aqueous composition including a bioactive agent. This soaking step may be performed in addition to co-precipitating a drug onto the surface of an implant as described above. Alternatively, loading a bioactive agent onto the implant surface by performing a soaking step may be suited to situations where the implant was manufactured under conditions that would destabilize, degrade, or otherwise adversely affect the function of the drug. The soaking step may be performed without limitation with regard to strength, composition, pH or temperature of the soaking solution. Charging the implant by performing a soaking step may be suited to situation where activation of the drug must be performed under conditions that are adverse to the precipitation and/or formation of crystalline HAp on implant surfaces.

### EXAMPLES

The following will serve to illustrate, by way of one or more examples, systems and methods for inhibiting, reducing or otherwise disrupting prolactin signaling in pain neurons according to some embodiments. The examples below are non-limiting and are intended to be merely representative of various aspects and features of certain embodiments. Although methods and materials similar or equivalent to those described herein may be used in the application or testing of the present embodiments, suitable methods and materials are described below.

### Example 1

Formation of a Hardened Calcium Phosphate Cement Article

Porous calcium phosphate cement coupons were made by the following procedure. An injectable paste of calcium phosphate cement was prepared by mixing 0.6 g of whiskered TTCP powder (made according to the procedures set forth in U.S. Patent Appl. Publ. No. 2004/0003757) with concentrated (NH₄)₂HPO₄ solution in water at a liquid to solid ratio of 0.3 for 1 min. The paste was then thoroughly mixed with a mixture (1:1) of NaCl and KCl salt particles (pore forming powder). The amount of salt mixed with the paste was equal to the dry weight of the salt used to make the paste. The resulting paste mixture was filled into a cylindrical stainless steel mould having a diameter of 12 mm and compressed with a gradually increased pressure up to about 45 MPa and the cement was allowed to harden. The hardened material was immersed in distilled water at 37° C. for 48 hours and dried in air for 24 hours.

Formation of a Nanocrystalline HAp Layer in Implant Surfaces

### Example 2

The dried material made in Example 1 was immersed in Hank's balanced salt solution 1×, HyQ®HBSS cell culture reagents without Phenol Red, 0.1 μm sterile filtered; HyClone, (Logan, Utah) for 3 days, rinsed with distilled water and air dried for 24 hours.

### Example 3

The dried material made in Example 1 was immersed in phosphate buffered saline (PBS) for 5 days, rinsed with distilled water and then dried in air for 24 hours.

The hardened CPC discs made in Examples 2 and 3 were gold coated and the surface morphology of nanocrystalline HAp was examined using scanning electron microscopy. Representative images are shown in FIG. 1A (samples incubated in HBSS for 3 days), and FIG. 1B (sample incubated in PBS for 5 days). As shown in FIG. 1, a nanocrystalline nanoporous mineral layer was formed after the surface modification with either HBSS or PBS

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes may be made in the elements described herein without departing from the spirit and scope of the invention as described in the following claims. In addition, it is to be understood that features described herein independently may, in certain embodiments, be combined.

1. A bone implant comprising:
one or more porous components;
a load bearing component surrounding at least a portion of a porous component, wherein a load bearing component comprises one or more protrusions positioned on at least one surface of the load bearing component;
wherein the load bearing component and at least one of the porous components comprise calcium phosphate, and wherein a porosity of at least one porous component is greater than a porosity of the load bearing component.

2-7. (canceled)

8. The implant of claim 1, wherein the load bearing component comprises one or more apertures extending through the load bearing component, wherein at least one aperture is at least partially filled with a porous component.

9. The implant of claim 1, wherein the load bearing component comprises one or more channels formed on a surface of the load bearing component.

10-14. (canceled)

15. The implant of claim 1, wherein at least one protrusion is configured to engage an endplate of a vertebra.

16. (canceled)

17. (canceled)

18. The implant of claim 1, wherein a first protrusion is positioned on a first surface of the load bearing component, and wherein a second protrusion is positioned on a second surface of the load bearing component, opposite the first surface.

19. The implant of claim 18, wherein two protrusions are positioned on the first surface, and wherein at least one protrusion is positioned on the second surface.

20-25. (canceled)

26. The implant of claim 1, wherein the implant has a shape which, when the implant is inserted in a spine, helps to maintain a natural lordosis of the spine.

27-35. (canceled)

36. A bone implant comprising:
one or more porous components, wherein at least one porous component comprises one or more openings extending through the porous component;
a load bearing component surrounding at least a portion of a porous component;
wherein part of at least one porous component extends beyond a surface of the load bearing component, wherein the load bearing component and at least one of the porous components comprise calcium phosphate, and wherein a porosity of at least one porous component is greater than a porosity of the load bearing component.

37. The implant of claim 36, further comprising one or more additives positioned in at least one opening in a porous component.

38. The implant of claim 36, wherein the load bearing component comprises a first surface and a second surface opposite the first surface, and wherein part of at least one porous component extends beyond the first surface of the load bearing component and the second surface of the load bearing component.

39-44. (canceled)

45. The implant of claim 36, wherein the load bearing component comprises one or more apertures extending through the load bearing component, wherein at least one aperture is at least partially filled with a porous component.

46. The implant of claim 36, wherein the load bearing component comprises one or more channels formed on a surface of the load bearing component.

47-51. (canceled)

52. The implant of claim 36, wherein at least one protrusion is configured to engage an endplate of a vertebra.

53. (canceled)

54. (canceled)

55. The implant of claim 36, wherein a first protrusion is positioned on a first surface of the load bearing component, and wherein a second protrusion is positioned on a second surface of the load bearing component, opposite the first surface.

56-64. (canceled)

65. A bone implant comprising:
a load bearing component;
one or more porous components coupled to an outer surface of the load bearing component;
wherein the load bearing component and at least one of the porous components comprise calcium phosphate, and wherein a porosity of a porous component is greater than a porosity of the load bearing component.

66-91. (canceled)
